(19) United States
(12) Patent Application Publication
(10) Pub. No.: US 2005/0059869 A1
(43) Pub. Date: Mar. 17, 2005

(54) Physiological monitoring system and improved sensor device

(76) Inventors: John Scharf, Oldsmar, FL (US); Scot Johnson, Tampa, FL (US)

Correspondence Address: PATENTMETRIX, 14252 CULVER DR. BOX 914, IRVINE, CA, 92604, US

(21) Appl. No.: 10/662,702

(22) Filed: Sep. 15, 2003

(51) Int. Cl. A61B 5/00

(52) U.S. Cl. 600/340; 600/344

(57) ABSTRACT

The present invention is directed toward a system for monitoring a region of a person to determine a plurality of physiological characteristics, including blood oxygenation levels, blood gases, respiratory rates, and pulse rates. The monitored region includes at least a portion of a dermal layer extending over anywhere on the chin, including at least one of the subject's mandible, symphysis, mental protuberance, or incisive fossa. The system comprises a sensor having at least one light emitting source and at least one detector. Preferably, the sensor is positioned on the region being monitored and is secured to the region being monitored by a securing means. Optionally, the securing means comprises a strap that is adjustable and in physical communication with the housing. In one embodiment, the strap is attached to an apparatus and the apparatus is capable of being secured to a head of the subject. The apparatus can be attached to a helmet and used for at least one of a military, sporting, construction, security, policing, or firefighting application.

### FIELD OF THE INVENTION

The present invention relates to a method and apparatus for the placement and use of a physiological sensor. More specifically, the present invention is a method and system for monitoring blood oxygenation (SpO₂) levels with an improved sensor configuration, to achieve greater accuracy.

### BACKGROUND OF THE INVENTION

The blood oxygenation level (SPO₂), other blood gases, and the respiratory rate of patients must often be monitored either periodically or continuously. Pulse oximetry is used to monitor the physiological conditions of patients, including but not limited to, the oxygen saturation of hemoglobin (SpO₂) in arterial blood and pulse rate. Pulse oximeters are now standard monitors that provide clinicians with a noninvasive indication of patients' cardiopulmonary status.

Oximeter sensors transmit light through blood-perfused tissue, such as a finger or an ear, and photo-electrically sense the absorption of light in the tissue. A wide variety of oximeter sensors are available from a number of manufacturers, including transmission type sensors, quasi transmission type sensors and reflectance type sensors. The light passed through the tissue has a wavelength that, when absorbed by the blood, is representative of the physiological condition being measured. Data received by the sensor is then communicated to a processing unit that presents the measured characteristics to a health care provider, such as a doctor, nurse, technician, or any caregiver.

Limitations of pulse oximetry include sensitivity to high levels of optical or electric interference, errors due to high concentrations of dysfunctional hemoglobins (methemoglobin or carboxyhemoglobin) or interference from intravascular dyes (such as methylene blue). Other possible limiting agents could be low perfusion states, artificial detection barriers such as nail coverings with finger probes, and the inability to quantify the degree of hypoxemia present. It has also been observed that perfusion levels are in direct proportion to the amount of blood capillary loops present in the skin region where the sensor is placed.

Conventional pulse oximeters have been used in critical care, anesthesia and post anesthesia care units, and home care. Such conventional pulse oximeters consist of a sensor, operating in combination with an oximeter unit that displays waveforms, oxygen saturation levels (SPO₂), the pulse rate, a perfusion index value, or other values. The perfusion index (PI) is an indication of the quality of patients' perfusion at the sensor site. To date, the sensors have been placed on certain, well-defined peripheral tissue beds, such as a finger, or an ear lobe, referred to herein as conventional skin regions.

However, these sensor and detection devices are limited in their utility to the field of monitoring clinical health parameters only. Currently the sensors are used in human body where there are a relatively low number of capillary loops per mm² of skin surface. The number of capillary loops on a defined area of the skin has a considerable effect on the accuracy of the sensor readings.

TABLE 1
Average Number of Capillary Loops on Conventional Skin Regions.

| Conventional Skin | Average Number of Capillary |
|---|---|
| Region | Loops (per mm²) |
| Scalp | 128 |
| Forehead | 145 |
| Nose | 100 |
| Lips | 130 |
| Chin | 158/149 |
| Ear | 38 |
| Upper Neck | 113 |
| Shoulder | 27 |
| Hand | 20-70 |
| Thigh | 29 |
| Lower Leg | 41 |
| Foot | 41 |

Attempts at improving pulse oximetry systems have largely focused on the improvement of algorithms used to calculate SpO₂ levels in blood; methods and systems for reducing noise generated due to motion or other artifacts; the shape and size of sensors used for measuring physiological parameters in blood; the sensitivity of sensors or detectors to pulsating components of blood; the amount or type of light used for the purpose of detection; or the sensor and pulse oximeter electronics and software used to measure the electrical signal at the sensor and convert the signal into clinically relevant information.

U.S. Pat. No. 6,343,223 discloses a method and apparatus for improving blood perfusion by heating patients' skin and providing emitters and a detector which are offset from each other. However, its focus is to improve perfusion levels for greater accuracy by incorporating a heating device at the monitoring site.

U.S. Pat. No. 5,549,113 discloses a method for monitoring selected physiological parameters of a subject and alerting a caregiver at a remote location when an irregularity is detected. The primary objective of the method is to monitor health parameters remotely. This system does not address the possibility of monitoring specifically non-conventional skin regions using a pulse oximeter.

U.S. Pat. No. 6,393,311 relates to processing a detected signal at the sensor and particularly to processing measured signals to remove unwanted signal components caused by noise due to motion artifacts. However, it does not cover motion artifacts due to conventional human activities, such as talking or eating, while taking SpO₂ measurements.

U.S. Pat. No. 5,817,008 describes an opto-electronic pulse oximetry system, which physically conforms to a body portion of a patient, such as a finger, and provides an apparatus for firm pressing engagement between the sensor and patient's body portion. This invention is limited to applications on conventional skin regions.

U.S. Pat. No. 5,421,329 discloses a SpO₂ measuring sensor with a light source optimized for low oxygen saturation changes and for maximizing the immunity to perturbation induced artifact. The primary objective of this invention is to devise a system that optimizes the light wavelengths during low perfusions and motion. However, it does not disclose information about any location where the sensor could be placed so as to have high perfusion levels under any condition.

U.S. Pat. No. 6,018,673 relates to optical sensing mechanisms for determining physiological characteristics in the presence of motion. U.S. Pat. No. 5,596,986 discloses a non-invasive blood oximeter that utilizes the principle of backscattered light to measure parameters related to blood oxygen content. U.S. Pat. No. 5,431,159 discloses a non-invasive pulse oximetry method wherein the red and infra red light coming from the tissue is sensed, in order to obtain frequency multiplexed information, as to the absorption of the said light by that tissue. It also includes a method for filtering the information obtained.

U.S. Pat. No. 5,437,275 discloses a pulse oximetry sensor with a housing that can be readily pre-assembled and which can be disposed of after a single use. U.S. Pat. No. 6,416,471 is directed to a disposable multi parameter sensor band, for measuring patient vital signs and transmitting the measured vital signs data to a remote monitoring location over a telecommunication link. This invention discloses conventional blood oxygenation sensors placed on the finger, wrist, or ear, which could provide data through a wire or wireless link to the sensor band.

All of the aforementioned patents are hereby incorporated by reference.

Despite existing inventions in the field of measuring physiological characteristics, conventional methods and systems do not effectively and accurately conduct pulse oximetry and related physiological measurements and monitoring at non-conventional skin region(s), particularly those regions that are rich in capillary loops and thus have high blood perfusion levels. Accordingly, existing embodiments fail to disclose appropriate physical configurations for sensing apparatuses, including housing type, shape, size, or other physical parameters, related to pulse oximetry sensors, for use in non-conventional skin regions.

Accordingly, there is a need for an improved sensor apparatus that could be applied to non-conventional skin regions, particularly those regions having high number of capillary loops per mm², thus improving the reliability of the readings obtained. There is also a need to incorporate improved software or algorithms for eliminating noise due to motion, or any other artifacts, unique to that non-conventional skin region. Moreover, there is a need for an apparatus that can be applied to the geometry of the non-conventional skin regions. Furthermore, there is a need to develop sensing devices that can be readily applied in settings outside a health care environment, such as a military or exercise-training environment.

### SUMMARY OF THE INVENTION

The present invention is directed toward a system for monitoring a region of a person, patient, living entity, or any type of subject to determine a plurality of physiological characteristics, including at least one of blood oxygenation level, blood gases, respiratory rate, and pulse rate. The monitored region includes at least a portion of a dermal layer extending over anywhere on the chin, including at least one of the subject's mandible, symphysis, mental protuberance, or incisive fossa. The system comprises a sensor having at least one light emitting source and at least one detector. Preferably, the sensor is positioned on the region being monitored and is secured to the region being monitored by a securing means.

Optionally, the system is a reflectance type sensor that comprises a housing with an edge region where the detector is attached to the housing and where the light emitting source is also attached to the housing and substantially adjacent to the detector. Preferably, the edge region is curved and substantially conforms to a contour of the region being monitored.

Optionally, the securing means comprises a strap that is adjustable and in physical communication with the housing. In one embodiment, the strap is in physical communication with an apparatus and the apparatus is capable of being secured to the head of a subject. The apparatus can be attached to a helmet and used for at least one of a military, sporting, construction, security, policing, or firefighting application.

Optionally, the system is a transmission type sensor that comprises a housing attached to the detector and a housing attached to the light emitting source. In one embodiment, the detector and light emitting source are positioned to permit light emitted from the light emitting source to pass into the region being monitored and out to the detector. In another embodiment, the light emitting source and the detector are positioned with the region to be monitored juxtaposed in between the light emitting source and the detector.

Optionally, the securing means includes an adhesive layer on the detector housing and on the light emitting source housing.

Optionally, the securing means comprises a first strap in physical communication with the detector housing and a second strap in physical communication with the light emitting housing. In one embodiment, the first and second straps are two separate straps positioned substantially opposite each other relative to the region being monitored. In another embodiment, the first and second straps are integrally formed into a singular structure. In another embodiment, the first and second straps are adjustable. In another embodiment, the first strap and second strap are in physical communication with an apparatus and the apparatus is capable of being secured to a head of the subject. The apparatus can be attached to a helmet and used for at least one of a military, sporting, construction, security, policing, and firefighting application.

The present invention is also directed toward a method for monitoring a region of a subject to determine a plurality of physiological characteristics of the subject wherein the region includes at least a portion of a dermal layer extending over anywhere on the chin, including at least one of the subject's mandible, symphysis, mental protuberance, or incisive fossa. The method comprises the steps of securing a sensor having at least one light emitting source and at least one detector to the region being monitored, emitting light from the light emitting source, where the light emitting source is positioned proximate to the region being monitored, and detecting light from the surface of the region being monitored using the detector, where the detector is proximate to the region being monitored.

The present invention is also directed toward an apparatus for monitoring a dermal region of a subject's head, the region at least partially covering anywhere on the chin, including at least one of a mandible, symphysis, mental protuberance, or incisive fossa, to determine a plurality of physiological characteristics wherein the apparatus is securable to the head of the subject. The apparatus comprises a plurality of straps and at least one sensor having at least one light emitting source and at least one detector attached to at least one of the straps.

The present invention is also directed toward a non-invasive, electro-optical sensor for removable attachment to a dermal layer of a person wherein the sensor is used to measure physiological characteristics of the person and the dermal layer covers anywhere on the chin, including at least one of a mandible, symphysis, mental protuberance, or incisive fossa of the subject. The sensor comprises a support structure having at least one substantially planar surface, a light emitting source having an emission surface where the emission surface is positioned in the planar surface and exposed to an external environment, a detector having a detection surface where the detection surface is positioned in a planar surface and is exposed to an external environment, and a curved edge region where the curved edge region substantially conforms to a contour of the dermal layer. In one embodiment, the sensor further comprises a divider positioned between the light emitting source and the detector.

These embodiments and other embodiments are further described in reference to the drawings and detailed description provided herein.

### BRIEF DESCRIPTION OF THE DRAWINGS

These and other features and advantages of the present invention will be appreciated, as they become better understood by reference to the following description when considered in connection with the accompanying drawings:

FIG. 1 is a graph depicting the number of capillary loops per mm² of the skin surface in different regions;

FIG. 2 provides an exemplary embodiment of an I-shape reflectance sensor;

FIGS. 3a-3d depict a plurality of perspectives of an embodiment of a reflectance-C sensor;

FIGS. 4a-4b depict a base and top view, respectively, of one embodiment of a suction reflectance sensor;

FIG. 5 depicts one embodiment of a multisite-Y sensor;

FIG. 6 diagrams a graphical approximation of an exemplary chin geometry;

FIG. 7 depicts one embodiment of a helmet and chin-strap apparatus of the present invention;

FIG. 8 depicts another embodiment of a helmet and chin-strap apparatus of the present invention;

FIG. 9 depicts one embodiment of the present invention;

FIG. 10 depicts another embodiment of the present invention;

FIG. 11 depicts an exemplary method for placing one embodiment of a sensor for the present invention;

FIG. 12 depicts one embodiment of a chin-strap and an exemplary method for fastening it appropriately; and

FIG. 13 depicts an exemplary set of graphical readings obtained by using a multisite-Y sensor on a chin region.

### DETAILED DESCRIPTION OF THE INVENTION

The present invention is directed toward improving and expanding how physiological characteristics are measured by positioning novel sensing devices in non-conventional skin regions, particularly those regions having high levels of blood perfusion. While techniques for efficient and accurate measurement of blood oxygenation levels are known generally, they have not been effectively applied to certain non-conventional skin regions, such as the chin of a person. The chin is an appropriate sensing region because it has a relatively high number of capillary loops per mm² of the skin as compared to the other regions, such as digits or ear lobes.

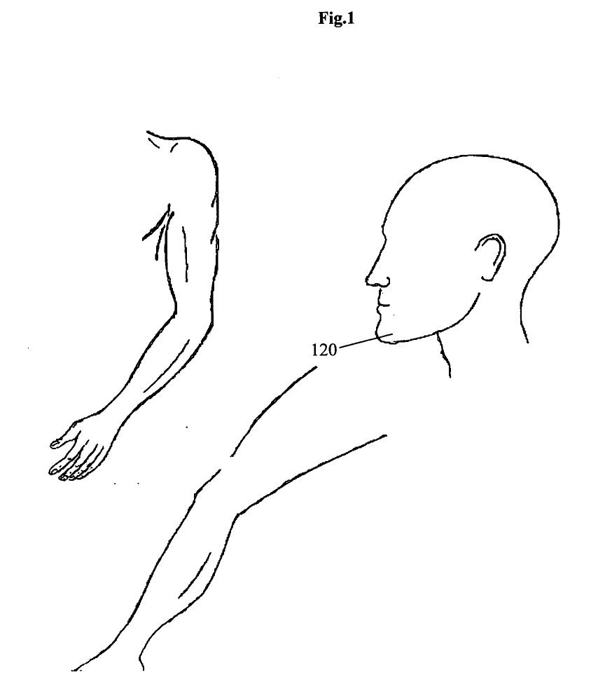

Referring to FIG. 1, it is observed that conventional skin regions have relatively low number of blood capillary loops per mm² 110 of the skin surface. For example, the nose has 100 blood capillary loops per mm², while the ear has 38 blood capillary loops per mm². Alternatively, there are approximately 149 to 158 blood capillary loops per mm², within the chin region 120 of a human patient.

The human chin region 120 may be defined as the central forward portion of the lower jaw, consisting of a horseshoe-like curved portion on the sides that unite the face and the neck, including the region comprising the mandible, symphysis, mental protuberance, and incisive fossa. In one embodiment of the present invention, the monitored region includes at least a portion of the dermal layer extending over anywhere on the chin region, including the mandible, symphysis, mental protuberance, and incisive fossa areas of the patient's body.

One embodiment of the present invention is to obtain high quality plethysmography waveform data by monitoring the chin region 120 using either reflectance or quasi-transmission based pulse oximetry sensors. While a plurality of different sensors can be used, and the present invention is not limited to a specific type of sensor, the present invention shall be described in relation to three forms of reflectance sensors and one form of quasi-transmission sensor. A transmission oximeter sensor operates by detecting light transmitted by a source through an object to be sensed. In one embodiment the light source comprises two light-emitting diodes (LEDs), emitting light of different wavelengths placed on one side of the target object. At least one sensor capable of detecting the emitted wavelengths is placed on the other side of the target object, opposite the light source. Alternatively, a reflectance oximeter sensor operates by detecting light reflected by the object to be sensed. The light source comprises two light emitting diodes (LEDs) of different wavelengths placed on one side of the target object and at least one sensor sensitive to the emitted wavelengths placed on the same side as the light-emitting source.

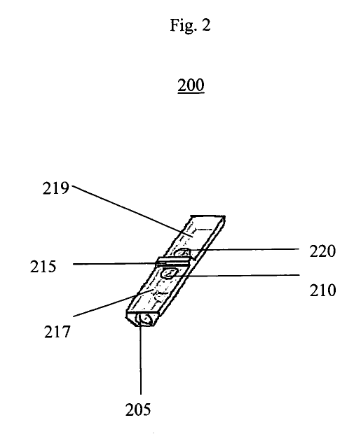

Referring to FIG. 2, an I-shaped reflectance sensor 200 comprises an emitter 210 and a detector 220 placed opposite and in line with each other. The sensor further comprises a hinge 215 operative to enable the protrusions 217, 219 within which the emitter and detector are embedded to fold and encompass a sensing region. At least one protrusion 217 preferably comprises a cavity 205 for providing an interface with the sensor monitor. The interface may be provided in the form of a wire or wireless connection.

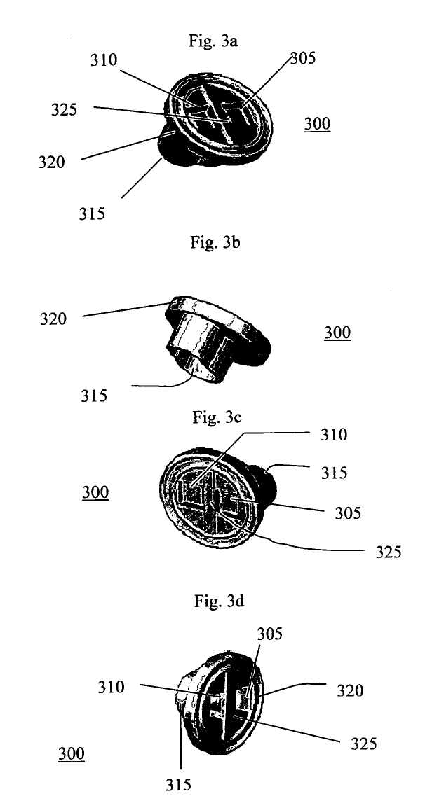

Referring to FIGS. 3a-3d, the reflectance C-sensor 300 comprises an emitter 305 and a detector 310 mounted within a circular base 320. The emitter 305 and detector 310 may be separated by a platform, such as a raised, straight, or narrow platform 325, acting as a barrier between the two. The other side of the circular base 320 houses a cavity 315, formed by protruding walls, attached to the circular base 320, that are shaped for providing a wired or wireless interface with an oximeter monitor. The configuration of the reflectance C-sensor is such that it can be rotated to monitor a region at an appropriate angle. This feature enables the sensor to be attached suitably according to the shape of the skin region over which it is placed. It also permits relatively easy positioning of the reflectance C-sensor where the readings can be obtained more reliably.

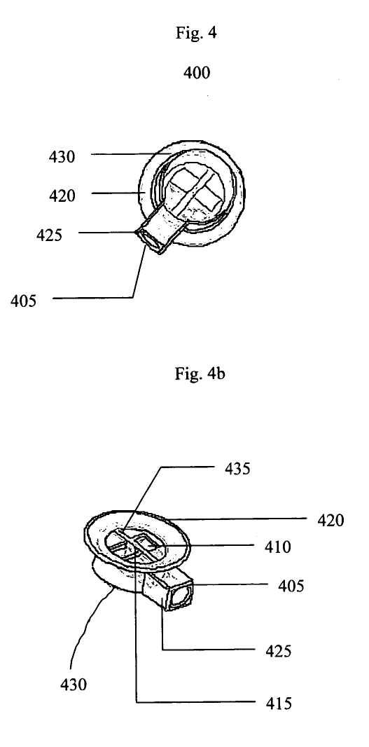

Referring to FIGS. 4a-4b, another embodiment of the present invention is illustrated in the form of a suction reflectance sensor 400. The sensor 400 comprises an emitter 410 and a detector 415 mounted over a base 430, preferably circular, and separated by a raised narrow platform 435. In one embodiment, the walls of the mechanical housing are inclined, to which the emitter 410 and detector 415 are attached, thereby increasing the amount of light that transverses the dermis from the LED to the photodiode. An edge region 420 physically attached to the base 430, surrounds the emitter 410 and detector 415. The border, edge, or outer area 420 is preferably designed to block ambient light interference by providing a shield from external light sources, as well as a suction attachment to the region being monitored. The border 420 is preferably curved, more preferably parabolic in shape, to effectively provide contact with a curved region, such as the chin region. A configuration with the curved border in physical contact with the skin region can be rotated to monitor the region at any angle with respect to where the emitters and detectors are located. The base 430 may house a cavity 405 formed by walls 425 protruding from the base 430. The cavity 405 is preferably used to house a mechanism for interfacing the sensor with the oximeter monitor. This mechanism may be in the form of a wire or wireless connection.

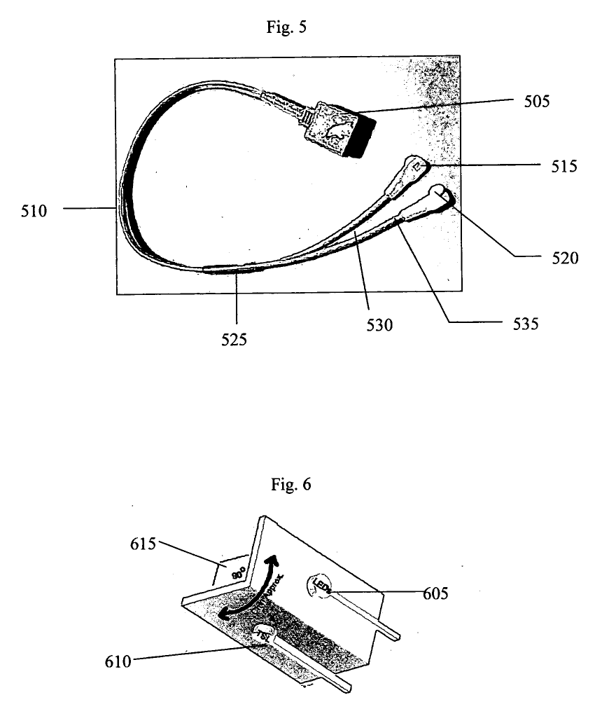

Referring to FIG. 5, the quasi-transmission sensor 500 comprises an emitter 515 and a detector 520 housed separately in compartments integrally formed with separate cabling encompassing electrical connections 530, 535. The emitter 515 is connected to a wire 530 and detector 520 is connected to a separate wire 535. The two wires 530, 535 may be joined 525 after a certain length, with a suitable adhesive, tape or any other binding mechanism. The joined probes 510 are then connected to a plug 505, which may be used for interfacing the sensor probe 500 with the oximeter monitor.

Although it is possible to use any of the reflectance, transmission or quasi-transmission type of sensors, unconventional skin regions offer certain limitations due to their geometry. For example, as shown in the exemplary graphical depiction of FIG. 6, the chin of human beings has an irregular geometry. For the unique shape of the chin, effective monitoring for a transmission type of sensor requires the placement of the emitter 605 and detector 610 at an alignment of approximately 90 degrees 615 relative to each other. This relative configuration is preferred for effective monitoring of the chin region and is effective in eliminating channeling of light as is problematic in reflectance-type sensors.

The present invention further comprises a means to secure a sensor, including any of the sensors described above, to a skin region, such as the chin region. In one embodiment, at least one light emitting source is attached, embedded, or otherwise incorporated into a first housing, and at least one detector is attached, embedded, or otherwise incorporated into a second housing. As known to those of ordinary skill in the art, the emitting surface of the light emitting source and receiving surface of the detector are exposed to an external environment through an application surface of the first and second housings. The securing means includes an adhesive layer on the application surface of the first and second housings. The adhesive type may be any type of adhesive known to those skilled in the art.

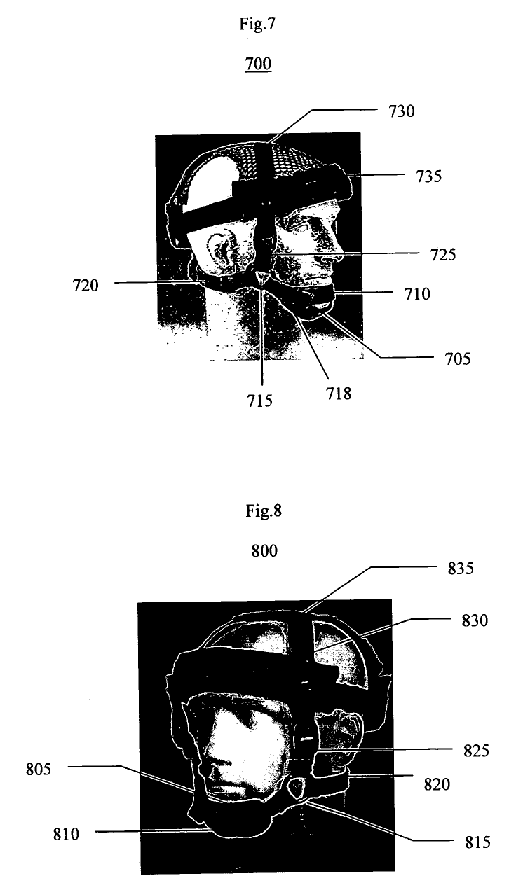

In one embodiment, the emitter and detector components are incorporated into a chin strap apparatus that may be integrally formed with a protective head apparatus for sports, training, firefighting, construction, policing, security, or military applications. Referring to FIG. 7, the chin strap apparatus 700 may comprise two sections 710, 705, extending over the front and lower portions of the chin respectively, within which are integrally formed an emitter and a detector, thereby enabling quasi-transmission mode monitoring. The strap 710, 705 components are securely attached through an attachment mechanism 718, such as Velcro, a button snap, or other means and physically integrated with a head apparatus through a triangular ring 715. Although shown as a strip of material, the term strap can refer to any physical structure used to act as a harness, holster, wrap, bandage or other securing means. The triangular ring 715 is in further physical communication with a neck strap 720, and a strap 725 connected to the suspension system 730 and headband 735.

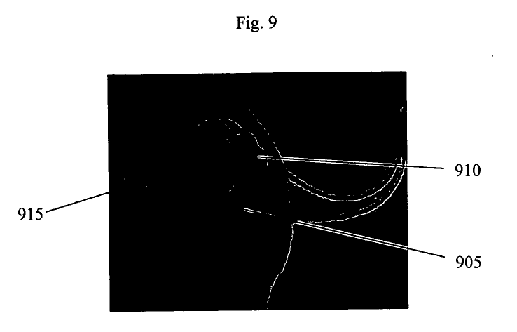

In this embodiment, an emitter emits light through the back of the front strap 710, is transmitted through the chin region, and detected by detectors having an obstructed optical communication, through the back of the back strap 705, with the monitored region. Referring to FIG. 9, an exemplary configuration of the above described embodiment is shown without the associated strap components. An emitter 910 emits light which is transmitted through a chin region 915 and received by detectors 905.

Referring to FIG. 8, another configuration of a head apparatus for sports, training, firefighting, construction, policing, security, or military applications is shown. A chin strap 810 is integrally formed with a cup 805 that accommodates sensor electronics. The chin strap 810 may be attached to a structure 815 that provides support to the neck pad 820 and the strap 825 connected to a suspension system 835 and headband 830.

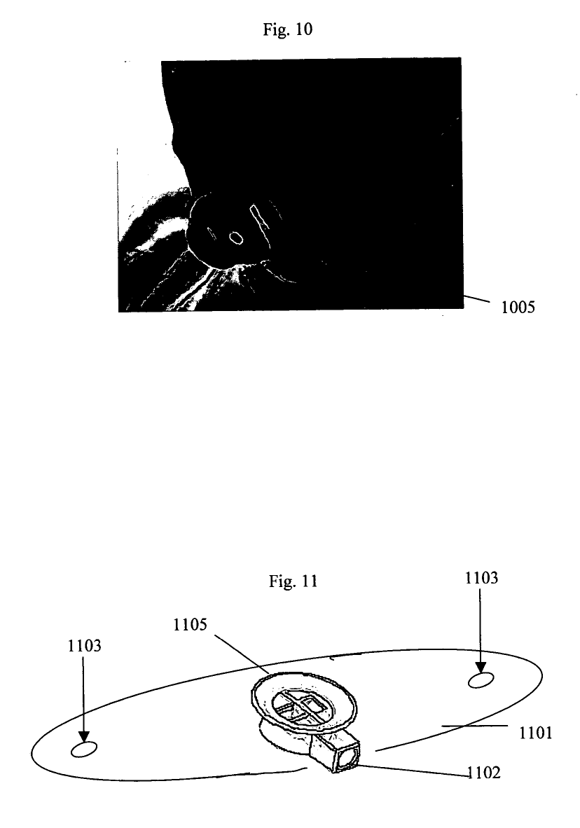

In one embodiment, the chin strap 810 and cup 805 preferably accommodates a C-reflectance type sensor. The sensor encompasses, and is in physical contact with, the region, such as the chin region, which is being monitored for physiological parameters. Referring to FIG. 10, an exemplary configuration of the above described embodiment is shown without the associated strap components. A reflectance type sensor 1005 comprises an emitter that emits light, which is reflected from a chin region, and detected by detectors. FIG. 10 illustrates the black-suction-reflectance sensor of the present invention and its location on the chin, for monitoring of physiological parameters. Any suitable reflectance sensor, as known to those of ordinary skill in the art, could potentially be secured into a chin strap and/or chin cup.

Integrating a sensor into a chin strap or chin cup assists with minimizing the degree of optical or electrical interference that may be caused by external conditions and, therefore, may minimize noise due to the exposure of detectors to ambient light. In one embodiment, the shape, material, color and depth of a chin cup is designed to maximize ambient light blockage while minimizing the overall size. Preferably, the shape of the chin cup comprises a sufficient volume to incorporate the sensor so that the emitter in the sensor directs its radiation to the correct location over the chin surface, and simultaneously, the detector is aligned with the emitter at a correct inclination so as to receive the reflections. The chin strap color is preferably black, which aids in blocking ambient light and the depth provides space for embedding the sensor apparatus. While the present invention has been discussed with reference to head apparatuses for sporting, training, and military applications, the present invention may be deployed in any kind of helmet chin strap configurations (for example single headband strap, single or double nylon chin straps, or any other chin-strap made from any material, shape, or size) or external to a head apparatus, comprising just a chin strap alone.

Another embodiment of the present invention, deployed external to a head apparatus, is depicted in FIG. 11. The sensor 1105 can be attached to a flexible oval shaped cloth 1101, with holes 1103 on both ends of the oval 1101. The plate 1101 could be made of any material, such as plastic or nylon, so as to be flexible as well as comfortable for the wearer, comparable to a surgical mask. The sensor 1105 is mounted within the oval plate 1101 with the help of an attachment mechanism, such as an adhesive. The two holes 1103 on either end may be used to secure the sensor to the sensing region, such as the chin region, by securing the structure to an existing apparatus, such as a plurality of straps through a pair of rivets (i.e. standard aluminum split rivets with stainless steel pins) or by passing elastic or string through the holes and passing it around the subject's head. The sensor should be placed in direct contact with the sensing region, such as the chin surface, which is then monitored to obtain a set of physiological parameters.

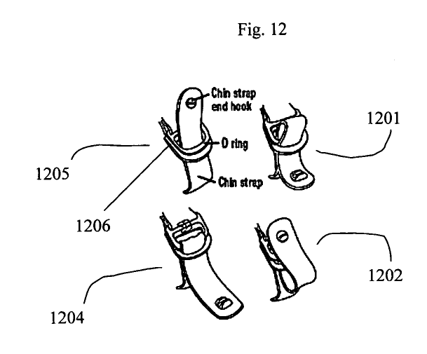

For a sensor apparatus that is embedded in a head apparatus and chin strap, it is preferred that the head apparatus be securely fastened to the skull of the wearer. An example for how to do the same is demonstrated with the help of a chin strap employing a D-ring system, shown in FIG. 12. If the strap 1205 is not substantially firmly pressed against the chin of the subject, the strap should be further secured through the D-rings 1206. To securely fasten the D-ring retention system 1206, ends of the chin strap should be threaded through the D-rings, as shown in FIG. 12, and pulled tight. Preferably, the chin strap end hook is clipped 1202 on to the D-ring. This secures the loose end of the chin strap after securely tightening the strap and avoids having the end portion of the chin-strap remain loose.

A typical pulse oximeter system consists of a probe connected to a probe interface circuit by means of a set of electrical conductors. The probe, which may be termed as the input device, consists of an exterior housing that applies the active elements of the probe to the chin, containing arterial blood flow that is to be monitored. Active elements of the probe contain red and IR LED light sources and a photodetector to convert transmitted or reflected light into an electrical signal. In order to distinguish between the light beam produced by the red and infrared LEDs, these LEDs are synchronously sampled. Any sampling technique could be used, including TDM, FDM, or any other.

The probe interface circuit, in a preferred embodiment, converts light to frequency signals via use of a TSL-230 series photodiode. Optionally, the probe interface circuit is employed to collect analog signals, which are converted to digital signals by means of appropriate data processing circuit(s). By implementing suitable processing steps, the circuit consequently computes the SpO₂ level of the blood, pulse rate, respiratory rate, level of perfusion, signal to noise ratio, among other physiological parameters. One processing step calculates the ratio of the normalized derivative (or logarithm) of the red intensity to the normalized derivative (or logarithm) of the infrared intensity. This calculation yields a constant that is indicative of the partial oxygenation of the hemoglobin in the arterial blood flow. It is then possible to calculate the pulsatility index and other parameters.

The data received by the probe interface circuit can include a fairly significant noise component which is caused by a number of sources including the introduction of ambient light into the housing, as mentioned above, artifacts due to talking and eating, and various sources of electrical noise. In a preferred embodiment, the present invention makes use of fast Fourier transform (FFT) as the primary digital signal processing technique. Optionally, the present invention could employ various filtering techniques, known to persons of ordinary skill in the art, to minimize the impact of noise on the SpO₂ and other parameters measured by the system.

An exemplary signal processing technique to boost the signal to noise ratio may be used to reduce noise due to motion artifacts at the chin, caused by talking or eating. This technique may also assist in dealing with difficulties that may be experienced due to signal levels generated by the sensor. Conventional filtering techniques such as low pass, band pass, high pass filtering, and multiple notch filtering can be used to remove noise signal components from the measured composite signal. However, these filtering techniques are effective only in removing DC noise components. The DC noise components are introduced due to the tissues of constant thickness within the chin, including, for example, bones, muscles, and skin. Other motion artifacts experienced by sensors placed at the chin region and that may be caused by talking or eating generates AC noise components.

A preferred embodiment of the present invention incorporates a method for eliminating AC noise signals. This can involve acquiring a segment of raw data, which may be both red and IR measured at the detector from the respective light sources, analyzing the data segment for dominant frequency components, determining the frequency component which represents a valid plethysmographic pulse, computing an average pulse based on the correct frequency component and repeating for new raw data segments. Any suitable software for computing the average pulse may be used according to the invention. This processing is performed for all candidate frequencies. The modified average pulse with the highest quality measure is selected then scaled to place each diastolic peak at the same signal level to allow the pulses to be appended to one another without discontinuity. This permits the average pulse to be available for further processing to obtain plethysmographic readings. The above methods may be repeated once another full heartbeat pulse of data is collected for both the red and IR signals. The new pulse of data is added to the red and IR segments, respectively, and the oldest pulse of data is removed.

Preferably, one embodiment for the above-described system is a motion artifact rejection circuit card with an input/output (I/O) device, a processor, and a memory for storing a computer programmed algorithm for motion artifact rejection. The processor may be a digital signal processor. The I/O device may be any circuitry that allows communication to and from external circuitry, for example, bus interface circuitry. The I/O device may include a circuit card edge connector for plugging into the SpO₂ monitor. The memory may be any solid-state electronic memory suitable for storing digital data. The motion artifact rejection card may be a part of the complete SpO₂ measurement system for eliminating artifacts due to talking or eating, in electrical signals and calculating and displaying physiological parameters.

The present invention also includes a processor and an output device. The output device may be a display device such as a cathode ray tube device, liquid crystal display, active matrix display, a PDA, a laptop, a mobile phone, or any other suitable device known to a person skilled in the art. Alternatively, the output device may be a printer for producing a permanent or written record, such as a laser printer, ink jet printer, thermal printer, dot matrix printer or any other suitable printer known to one of skill in the art. The storage device may be a disk drive, or any kind of solid-state electronic memory device suitable for storing digital data including, for example, computer code and measurement data.

The input and output could be interfaced via any data-transferring medium (a cable or any other). It is also possible to use a networked sensor that can communicate with the parameter display device via wireless means. Wireless embedded sensors combine sensing, computation, and communication into a single device. Various communication protocols may be deployed on ASICs that provide low-power implementations of these protocols such as found at 2.4. gHz, for example Bluetooth or WI-FI (802.11b). Other useful protocols include IEEE standards 802.11a and 802.11g, which are in the 5 gHz range.

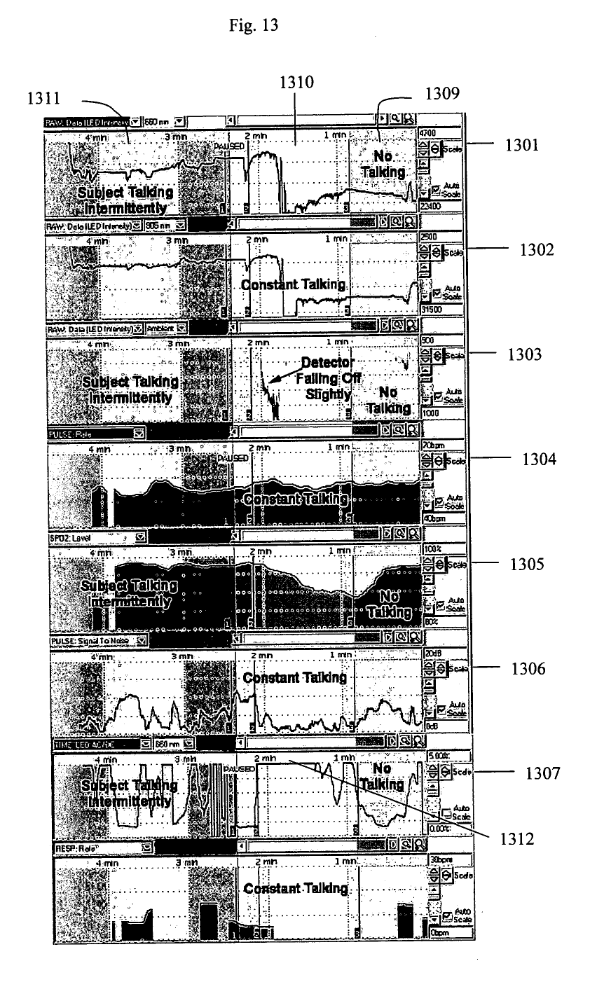

A plurality of parameters may be measured and calculated, as shown in FIG. 13. Referring to FIG. 13, the graphical forms of the various components of the signal produced by the light detector as a result of a light beam interacting with vascularized tissue are illustrated.

The light detector output signal consists of a large magnitude non-pulsatile component (DC component) and a small magnitude pulsatile component (AC component). The non-pulsatile component represents the light remaining after absorption due to a combination of venous blood flow, tissue, bone, and constant arterial blood flow while the pulsatile component is caused by light absorption due to pulsatile arterial blood flow that is to be measured. Because the LEDs are sampled in rapid succession, the data signals produced by the light detector consist of a plurality of sets of measurements, which may include the following:
1. Raw data 1301 reflecting the detected signal of light transmitted through, or reflected from, the monitored region;
2. Raw data 1302 reflecting a signal generated from ambient light interfacing with the detectors;
3. Pulse rate 1303, which is the number of heartbeats per minute (bpm). However, the pulse may also note information about the rhythm and strength of the heartbeat and whether the blood vessel feels hard or soft. An irregular rhythm, a weak pulse, or a hard blood vessel may indicate a medical condition that needs further evaluation. It is also an indication of dizziness, fainting, chest pain, or shortness of breath, fever, stress, an overactive thyroid gland (hyperthyroidism), anemia, stimulants (caffeine, amphetamines, decongestants, asthma medications, diet pills, and cigarettes), and various forms of heart disease.
4. SpO2 level 1304, which can be derived by applying a suitable algorithm to the detected raw data signals;
5. Signal to noise ratio 1305, where the signal represents the physiological parameter being measured and the noise is extraneous information received by the detector. This is commonly experienced in monitoring due to low perfusion or high motion;
6. LED AC to DC ratio 1306, which is an indication of blood perfusion levels; and
7. Respiratory rate 1308.

The above described information is obtained over a defined time period. In the illustrated charts, the defined time period comprises a one minute period, during which the subject is not talking 1309, a one minute period, during which the subject is constantly talking 1310 and a two to three minute period, during which the subject is talking intermittently 1311. As shown in FIG. 13, the blood perfusion levels remain at a significant value 1312, mostly above 1%, particularly when the subject is talking 1310, 1311. The raw data obtained for intensity of red 1301, infrared 1302 and ambient 1303 lights could be used to determine noise sources that can then be removed to obtain a more precise SpO₂ reading, as discussed earlier.

The head apparatuses, as explained and described above, could be effectively used to evaluate the health status of the army and other personnel during training and other physical and mental testing conditions (stress examination, lie detector test, physical training or test, military exercise, or any other test), by monitoring the chin. More importantly, the present invention could be employed to detect a casualty situation, or rather, triage a patient. The present invention, in a situation where the patient is semi-conscious, with little movement or talking, or even fully unconscious, critically ill, or dead, can relay vital information about the casualty to the local medic. The relay method can be through any communication mechanism known in the art, including through a networked sensor that can communicate with the parameter display device via wireless means. Various communication protocols may be deployed on ASICs that provide low-power implementations of these protocols such as found at 2.4. gHz, for example Bluetooth or WI-FI (802.11b). Other useful protocols include IEEE standards 802.11a and 802.11g, which are in the 5 gHz range. In an exemplary embodiment, the present invention is used by a field medic to triage wounded soldiers in the battlefield, determine which soldiers need immediate assistance, and identify certain soldiers as requiring no assistance, either because the soldiers are dead or healthy.

To evaluate the fitness of a subject, it is preferable to determine the ability of lungs to exchange oxygen and carbon dioxide, and the effectiveness of heart as a pump. It is also preferred to know whether the subject, who might be displaying normal cardiac function, is affected by COPD (Chronic Obstructive Pulmonary Disease). Oxygen content can be used as an indicator of lung diseases, such as emphysema and sarcoidosis.

Further, readings obtained in the present invention indicate the pulse and breathing rate of the subject and could be used to identify other health issues in the subject, such as high blood pressure (hypertension) or high blood pressure in the lungs (pulmonary hypertension) during virtual training exercises.

One of ordinary skill in the art would appreciate that the present invention can be expanded to many other applications. For example, the present invention can comprise an input device for a computer, which includes a mouthpiece and an interface for coupling the mouthpiece to the computer. The mouthpiece comprises a transmission, quasi transmission or reflectance type sensor for monitoring the chin, to obtain SpO₂ readings, as well as other physiological parameters, as discussed earlier. The computer is a mobile phone, a desktop, a laptop, a PDA, or any other data receiver and display device. The interface is either wire-based or wireless. In another embodiment, input to the computer may optionally comprise the parameters monitored by a SpO₂ probe, where the sensor for the probe operates as an attachment or is embodied within the input device. Such input devices include accessories worn by individuals that have a mouthpiece, headphone, or earphone. Individuals who wear devices, as defined above, may include physically challenged, astronauts, air-plane pilots, SCUBA divers, surgeons, construction workers, musicians, miners, among others.

The present invention may incorporated into a plurality of different apparatuses, including sports helmets, such as, helmets for hockey, baseball, football, biking, rafting, skiing, driving, and other forms of racing, further including helmets for construction workers, firefighters, miners, and others. The present invention is useful in monitoring the physiological parameters of a subject who is wearing the head apparatus, under normal or extreme, active or testing, conditions, particularly where the active and testing conditions relate to the activity of the wearer, such as in athletics, sports, firefighting, the military, security operations, or construction operations. In an alternative embodiment, face masks worn by fire-fighters, miners, soldiers, among others, may comprise the present invention to track asphyxiation conditions, which, if not monitored, may prove fatal.

Although this invention has been described with reference to particular embodiments, the invention is not limited to these described embodiments. Rather, it should be understood that the embodiments described herein are merely exemplary and that a person skilled in the art may make many variations and modifications without departing from the spirit and scope of the invention. All such variations and modifications are intended to be included within the scope of the invention as defined in the appended claims.

1. A system for monitoring a region of a subject to determine a plurality of physiological characteristics of said subject wherein said region includes at least a portion of a dermal layer extending over a chin region of said subject, said chin region including at least one of the subject's mandible, symphysis, mental protuberance, or incisive fossa, comprising:
a sensor having at least one light emitting source and at least one detector wherein said sensor is positioned on the region being monitored; and
a securing means to position said sensor to the region being monitored.

2. The system of claim 1 wherein the sensor is a reflectance type sensor.

3. The system of claim 2 wherein the sensor further comprises a housing having an edge region wherein said detector is attached to the housing and said light emitting source is attached to the housing and substantially adjacent to the detector.

4. The system of claim 3 wherein the edge region is curved.

5. The system of claim 4 wherein the curved region substantially conforms to a contour of the region being monitored.

6. The system of claim 1 wherein the detector is in data communication with a monitor.

7. The system of claim 6 wherein the monitor is an oximeter.

8. The system of claim 1 wherein the physiological characteristics includes at least one of blood oxygenation level, blood gases, respiratory rate, and pulse rate.

9. The system of claim 3 wherein the securing means comprises a strap.

10. The system of claim 9 wherein said strap is adjustable.

11. The system of claim 9 wherein said strap is in physical communication with the housing.

12. The system of claim 11 wherein said strap is in physical communication with an apparatus and wherein said apparatus is capable of being secured to a head of the subject.

13. The system of claim 12 wherein the apparatus is attached to a helmet.

14. The system of claim 12 wherein the apparatus is used for at least one of a military, sporting, construction, security, policing, and firefighting application.

15. The system of claim 1 wherein the sensor is a transmission type sensor.

16. The system of claim 15 wherein the sensor further comprising a housing attached to said detector and a housing attached to said light emitting source.

17. The system of claim 15 wherein said detector and said light emitting source are positioned to permit light emitted from said light emitting source to pass into said region being monitored and out to said detector.

18. The system of claim 15 wherein the light emitting source and the detector are positioned with said region to be monitored juxtaposed in between said light emitting source and said detector.

19. The system of claim 15 wherein the detector is in data communication with a monitor.

20. The system of claim 19 wherein the monitor is an oximeter.

21. The system of claim 16 wherein the securing means comprises an adhesive layer on said detector housing and on said light emitting source housing.

22. The system of claim 16 wherein the securing means comprises a first strap in physical communication with said detector housing and a second strap in physical communication with said light emitting housing.

23. The system of claim 22 wherein said first and second straps are two separate straps positioned substantially opposite each other relative to the region being monitored.

24. The system of claim 22 wherein said first and second straps are integrally formed into a singular structure.

25. The system of claim 22 wherein said first straps and second straps are adjustable.

26. The system of claim 22 wherein said first strap and second strap are in physical communication with an apparatus and wherein said apparatus is capable of being secured to a head of the subject.

27. The system of claim 26 wherein the apparatus is attached to a helmet.

28. The system of claim 26 wherein the apparatus is used for at least one of a military, sporting, construction, security, policing, and firefighting application.

29. The method for monitoring a region of a subject to determine a plurality of physiological characteristics of said subject wherein said region includes at least a portion of a dermal layer extending over a chin region of said subject, said chin region including at least one of the subject's mandible, symphysis, mental protuberance, or incisive fossa, comprising the steps of:
securing a sensor having at least one light emitting source and at least one detector to the region being monitored;
emitting light from said light emitting source wherein said light emitting source is positioned proximate to the region being monitored; and
detecting light from the surface of the region being monitored using the at least one detector, wherein said detector is proximate to the region being monitored.

30. The system of claim 29 wherein the sensor is a reflectance type sensor.

31. The system of claim 30 wherein the sensor further comprises a housing having an edge region wherein said detector is attached to the housing and said light emitting source is attached to the housing and substantially adjacent to the detector.

32. The system of claim 31 wherein the edge region is curved.

33. The system of claim 32 wherein the curved region substantially conforms to a contour of the region being monitored.

34. The system of claim 29 wherein the detector is in data communication with a monitor.

35. The system of claim 34 wherein the monitor is an oximeter.

36. The system of claim 29 wherein the physiological characteristics includes at least one of blood oxygenation level, blood gases, respiratory rate, and pulse rate.

37. The system of claim 31 wherein the securing means comprises a strap.

38. The system of claim 37 wherein said strap is adjustable.

39. The system of claim 37 wherein said strap is in physical communication with the housing.

40. The system of claim 39 wherein said strap is in physical communication with an apparatus and wherein said apparatus is capable of being secured to a head of the subject.

41. The system of claim 40 wherein the apparatus is attached to a helmet.

42. The system of claim 40 wherein the apparatus is used for at least one of a military, sporting, construction, security, policing, and firefighting application.

43. The system of claim 29 wherein the sensor is a transmission type sensor.

44. The system of claim 29 wherein the sensor further comprising a housing attached to said detector and a housing attached to said light emitting source.

45. The system of claim 29 wherein said detector and said light emitting source are positioned to permit light emitted from said light emitting source to pass into said region being monitored and out to said detector.

46. The system of claim 29 wherein the light emitting source and the detector are positioned with said region to be monitored juxtaposed in between said light emitting source and said detector.

47. The system of claim 29 wherein the detector is in data communication with a monitor.

48. The system of claim 47 wherein the monitor is an oximeter.

49. The system of claim 44 wherein the securing step is performed using an adhesive layer on said detector housing and on said light emitting source housing.

50. The system of claim 44 wherein the securing step is performed using a first strap in physical communication with said detector housing and a second strap in physical communication with said light emitting housing.

51. The system of claim 50 wherein said first and second straps are two separate straps positioned substantially opposite each other relative to the region being monitored.

52. The system of claim 50 wherein said first and second straps are integrally formed into a singular structure.

53. The system of claim 50 wherein said first straps and second straps are adjustable.

54. The system of claim 50 wherein said first strap and second strap are in physical communication with an apparatus and wherein said apparatus is capable of being secured to a head of the subject.

55. The system of claim 54 wherein the apparatus is attached to a helmet.

56. The system of claim 54 wherein the apparatus is used for at least one of a military, sporting, construction, security, policing, and firefighting application.

57. An apparatus for monitoring a dermal region of a subject, having a head, said region at least partially covering at least one of a mandible, symphysis, mental protuberance, or incisive fossa to determine a plurality of physiological characteristics wherein the apparatus is securable to the head of the subject, comprising:
a plurality of straps; and
at least one sensor having at least one light emitting source and at least one detector attached to at least one of said straps.

58. The apparatus of claim 57 wherein the sensor is a reflective type sensor.

59. The apparatus of claim 57 wherein the sensor is a tranmission type sensor.

60. A non-invasive, electro-optical sensor for removable attachment to a dermal layer of a person wherein the sensor is used to measure physiological characteristics of the person and the dermal layer covers at least one of a mandible, symphysis, mental protuberance, or incisive fossa of the subject, comprising:
a support structure having at least one substantially planar surface;
a light emitting source having an emission surface said emission surface being positioned in said planar surface and being exposed to an external environment;
a detector having a detection surface said detection surface being positioned in said planar surface and being exposed to an external environment; and
a curved edge region wherein said curved edge region substantially conforms to a contour of the dermal layer.

61. The sensor of claim 60 further comprising a divider positioned between said light emitting source and said detector.
